(19) United States
(12) Patent Application Publication
(10) Pub. No.: US 2015/0148757 A1
(43) Pub. Date: May 28, 2015

(54) Composite Cannula

(71) Applicant: Andreas Aeschlimann, Banyule (AU)

(72) Inventor: Andreas Aeschlimann, Banyule (AU)

(73) Assignee: SSB TECHNOLOGY PTY LTD, Scoresby, Victoria (AU)

(21) Appl. No.: 14/344,906

(22) PCT Filed: Sep. 12, 2012
(86) PCT No.: PCT/AU2012/001089

§ 371 Date: Jan. 18, 2015

(30) Foreign Application Priority Data
Sep. 13, 2011 (AU) 2011903736

(51) Int. Cl. A61B 17/34; B29C 47/02

(52) U.S. Cl. CPC A61B 17/3421; B29C 47/021
USPC 604/272; 29/428; 425/131.1

(57) ABSTRACT

The present invention provides a cannula comprising of a core needle and a sleeve, the core needle and sleeve spaced by conduit-forming projections. The projections may be aligned axially along the surface of the core needle or the inner surface of the sleeve. Engagement of the core needle with a sleeve forms a composite needle having one or multiple conduits for fluid transfer the length of the cannula. Engagement of the core needle and sleeve may be enhanced with different engagement means. Preferably the core needle and sleeve comprise of polymeric materials. The invention most advantageously can be produced without the need for a core pin in the formation of the core needle or sleeve. A composite needle may incorporate a sharp tip for penetration of materials and a side port for transfer of fluids. Alternatively, the composite needle may be blunt at the tip.

### FIELD OF INVENTION

This invention relates to the field of cannulae and injection needles, in particular polymeric cannulae and injection needles, and methods and apparatus for moulding of polymeric injection needles.

### RELATED APPLICATION

This application claims the priority and benefit of Australian Provisional Application No. 2011903736, filed on 13 Sep., 2011.

### BACKGROUND TO THE INVENTION

Many millions of injection needles are used for injecting substances every year. Injection needles used for medical purposes such as delivering therapeutic substances to subjects must be sterile to avoid contamination and as fine as possible to penetrate the skin to the site of delivery with minimal pain and damage. There must be a compromise between strength and gauge in needles to achieve both ends. Needles must be strong enough to penetrate and resilient to not break during penetration, which may lead to inadvertent injury during injection.

Metallic needles are well known in the art because of their strength properties. Metallic needles can be manufactured with a fine gauge but have adequate strength to penetrate tissues and not break. Recently, it has been shown that needles can be manufactured from polymeric materials as an alternative to metal needles. Stevens, Smith and Bartlett described a method for manufacturing polymeric hypodermic needles using gas-assisted injection moulding in U.S. Pat. No. 5,620,639, hereinafter referred to as the '639 patent, which is incorporated herein by reference. The method of the '639 patent is particularly useful for injection moulding polymeric needles for a single use and then disposal. Such polymeric needles are attractive because they are easy to manufacture inexpensively. Improvements of injection moulding methods and apparatus for polymeric needles were disclosed in patent applications published as WO2008/106728, WO2008/074065, and WO2010/071939. However, the compromise between the strength of a polymeric needle to resist breakage requiring a relatively large gauge means that very fine gauge polymeric needles may not be strong enough for many applications.

It is known in the art to minimise manufacturing expense by making single-use needles that comprise drawn stainless steel tubing fashioned to a point by grinding a number of facets onto the end of the tube. Such needles are simple to make but require penetration forces that can cause material damage or undue tissue injury because the ‘heel’ of the needle must cut its way through target material, which results in ‘coring’ from a slug of material being forced into the conduit as the needle penetrates the material.

‘Atraumatic needles’ have been designed to overcome the coring problem. These needles do not core material in penetrating and require less penetrative force because they incorporate side ports for transfer of substances. However, such atraumatic needles are expensive to manufacture. The expense of such needles restricts their use to special applications. What is needed is a needle that is simple and cheap to manufacture while being strong enough to penetrate material such as tissue to a desired depth for substance transfer with minimal damage to the material while penetrating the material. For example, there is a need for an inexpensive injection needle with a fine gauge that is strong enough to penetrate tissue without breaking or coring the tissue.

### BRIEF DESCRIPTION OF THE DRAWINGS

FIG. 1 shows an exploded perspective view of an embodiment of a needle the invention. FIG. 1a shows an embodiment of the invention as a composite injection needle. FIG. 1b shows the distal end of a needle with a blade at the tip. FIG. 1c shows the proximal end of a needle.

FIG. 2 shows a perspective view of a composite needle according to the invention. The sleeve (tubing) is shown in its assembled position. FIG. 2c shows the needle tip and the side ports.

FIG. 3a shows a longitudinal view of a needle according to the invention. FIG. 3b shows a cross section of the needle in FIG. 3a.

FIG. 4 shows in longitudinal section the engagement means for the sleeve. FIG. 4a shows the composite needle with engaged sleeve. FIG. 4b shows the engagement means at the distal end of the needle. FIG. 4c shows the engagement means at the tip portion of the needle.

FIG. 5 shows an embodiment of the invention with a blunt cannula tip.

FIG. 6 shows an exploded perspective view of an inserted cannula shaft . . . .

FIG. 7 shows an assembled view of an embodiment composite needle with an inserted core needle within a sleeve.

FIG. 8 shows in longitudinal section the engagement means for the sleeve and core needle.

FIG. 9 shows an exploded view of an embodiment composite needle where the sleeve (tubing) is an integrated part of the hub.

FIG. 10 shows in longitudinal section the engagement means of the hub with sleeve and the core needle.

FIG. 11 shows a double tip composite needle.

FIG. 12 shows alternative embodiments of spacer projections in a composite needle.

### SUMMARY OF THE INVENTION

Prior art teaching includes that small holes in cannulae for fluid transport, such as injection needles for pharmaceutical substances, may be formed using a mechanical core pin or, alternatively, by gas-assisted injection moulding as taught, for example, in the '639 patent, or mechanical coring methods. The present invention most advantageously overcomes problems with prior art methods in that that the fluid conduits are not formed with a core pin, allowing a less complex mould for polymer injection that eliminates the need for a small and fragile core pin in some applications. The core needle of the present invention can be produced with conventional polymer injection moulding techniques while enabling production of small gauge needles having an outer diameter with a lower range of about 0.2 mm. The polymeric material may be any suitable material for injection moulding such as the materials described in the '639 patent and further materials known in the art. The core needle and sleeve may be different polymers with differing properties suitable for achieving flexibility and strength. The sleeve requires resilience to slide over the core needle but not for strength. The core which contains the needle point requires strength to avoid tip deformation, breaking in use, including during penetrating and bending. The invention includes a method of manufacturing a composite cannula.

In one aspect, the invention provides a cannula comprising of a core needle having at least one channel for fluid transport and an engagement means; and a sleeve for engaging the core needle; wherein a sealed conduit for fluid transport is formed when the sleeve is engaged with the said core needle. Preferably the channel is defined by spacer projections from said core needle or said sleeve. More preferably, the spacer projections are axial spacers projecting from the core needle. Preferably the channel is longitudinal but it may also be formed from multiple projections, creating non-linear fluid flow pathways. Preferably, the cannula includes engagement means for engaging the sleeve with the core needle. Preferably, the engagement means comprises of a radial raiser bore connecting the longitude conduit with the needle hub. The cannula may include a tip with a sharp point. The cannula may include a tip with a sharp blade. The cannula may include an aperture or a plurality of apertures spaced from the tip end. The cannula may include a traumatic tip with side port. The cannula may have the sleeve integrated in the hub. Preferably at least one of the core needle or sleeve of the cannula comprises of polymeric material. Preferably, the cannula has an outside diameter of 0.2 mm or bigger.

In another aspect, the invention provides a mould for forming a cannula having at least one longitudinal channel in the peripheral surface of the cannula.

In another aspect, the invention provides apparatus to co-extrude a cannula as described herein. Preferably the apparatus combines two materials with different properties to form a cannula as described herein.

In another aspect, the invention provides a method of manufacturing a cannula for fluid injection the steps of forming a core needle having channels in the peripheral surface; forming a sleeve; and engaging the sleeve with the core needle. Preferably the engagement is frictional, but any other type of engagement may be used.

### DETAILED DESCRIPTION OF THE INVENTION AND MOST PREFERRED EMBODIMENTS

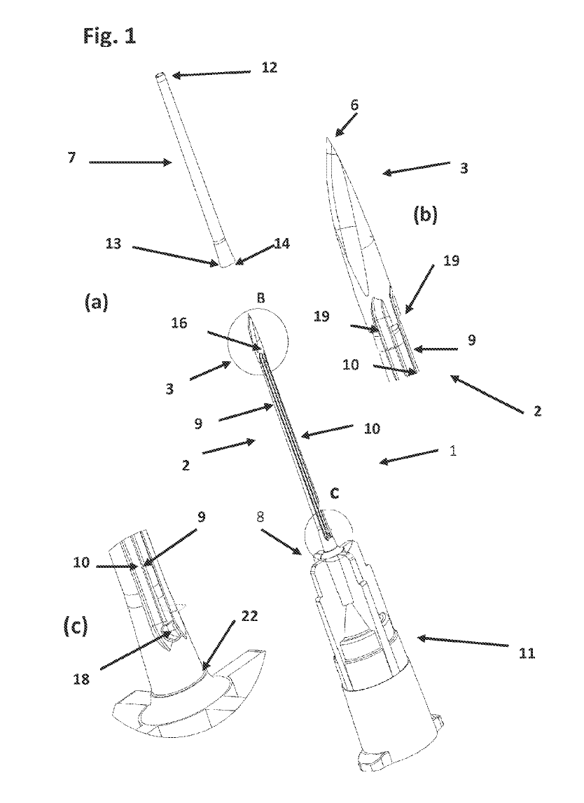

The objects of the invention are best understood with reference to the embodiments described herein and with reference to the figures. FIGS. 1 to 12 show embodiments of the invention. In the drawings, like features are indicated with the same numeral. It will be understood by those skilled in the art that figures in this disclosure are illustrative only and the invention is not limited to the embodiments shown in the figures but includes embodiments not illustrated but within the scope of the claims appended hereto.

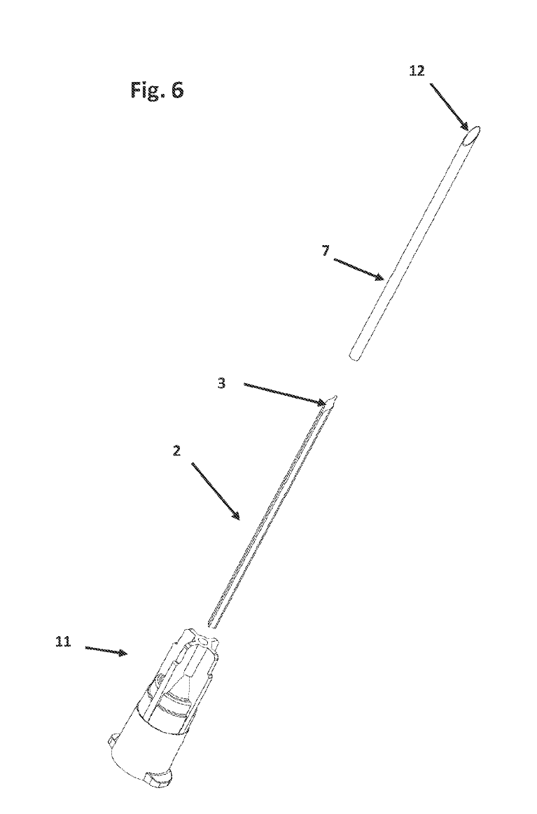

The invention provides apparatus for cannulae or needles for syringes. Preferably, the cannulae or needles comprise of injection moulded polymeric material. Whereas embodiments shown in the figures illustrate injection needles, the scope of the invention includes other types of cannulae for medical use, such as reconstitution needles or the like (or other uses such as in industrial applications). The invention also includes an apparatus for extruding or co-extruding a cannula For example, FIGS. 6-8 show a composite needle which could be manufactured by co-extrusion. In such an embodiment, the co-extruded core needle and needle sheet are engaged with the needle hub. The invention includes injection moulding apparatus for moulding cannulae, the apparatus incorporating at least one core needle having a least one channel for fluid flow and a sleeve for engaging the core needle for forming fluid conduits with the at least one channel or a plurality of channels. The invention includes embodiments with a single core needle having a plurality of conduits. The invention also includes multiple composite needles within a structure, such as a double composite needle incorporating two tips. The number of conduits may be one or more. Preferably, there are four conduits. Preferably the needle incorporates side ports for delivery of fluids at the injection site. The invention includes an atraumatic composite needle incorporating side ports.

Advantageously, the core needle and sleeve may comprise of any suitable material having suitable properties of strength and resilience, including but not limited to metals and polymers, or a combination thereof. Preferably both the core needle and sleeve comprise of polymeric materials. Most advantageously, the combination of a core needle within a sleeve provides a composite needle having adequate strength to be able to manufacture composite needles having relatively small gauges, such as about 0.2 mm outside diameter or larger.

The preferred embodiment of the invention will now be described with reference to the figures, which disclose a composite needle for a syringe, the needle incorporating a cutting blade at the tip. The most preferred embodiment includes axial or longitudinal channels formed by spacer projections projecting from a core needle and engaging with the inner surface of a sleeve. In this document, the words, axial and longitudinal are used interchangeably. Likewise, the words, spacer, and projection are similarly interchangeable.

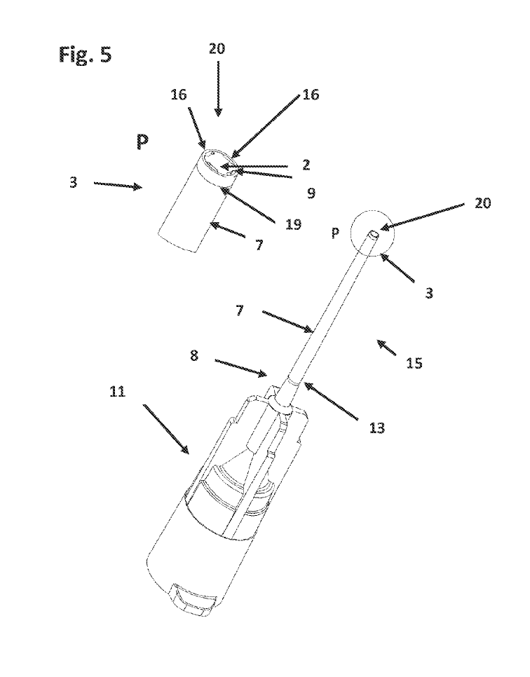

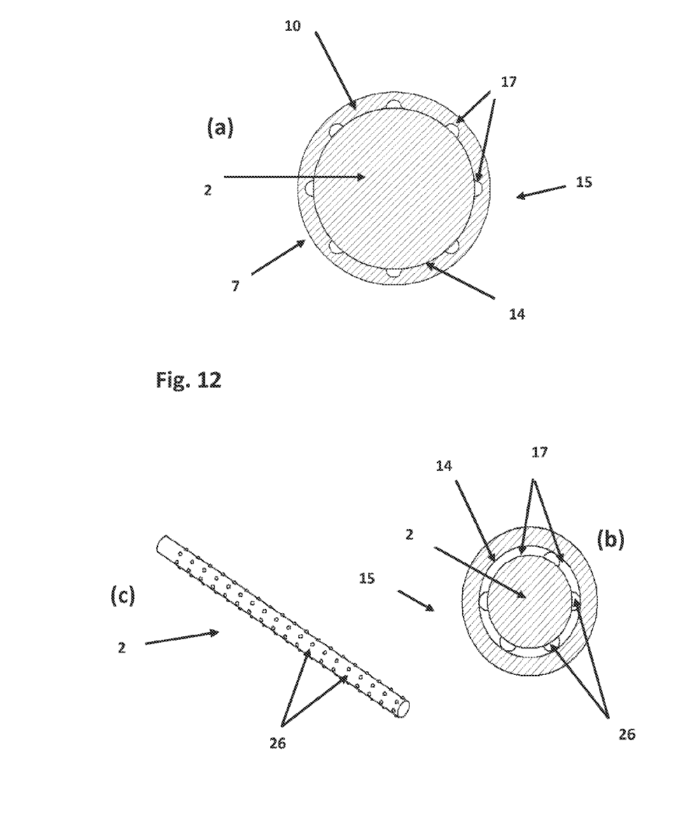

Many types of channels may be formed by other types of spacers. For example, bump-like spacers or projections may project from the surface of a core needle as illustrated in FIG. 12. Other types of channels may be formed or multiple channels may be formed within the scope of the invention. It will be understood that the invention includes other embodiments such as a cannula which may not incorporate a cutting blade or a sharp tip. Alternatively, the needle may not incorporate a cutting blade or sharp point at the tip, but may incorporate a simple aperture at a blunt tip as illustrated in FIG. 5, or other tip configurations.

FIG. 1 shows in side perspective an exploded cannula 1 having a hub 11, a core needle 2, and a sleeve 7. The sleeve 7 forms conduits for fluid transport along channels 9 formed by the inner surface 14 of the sleeve 7 and axial projections 10 of the core needle 2 parallel with the longitudinal axis.

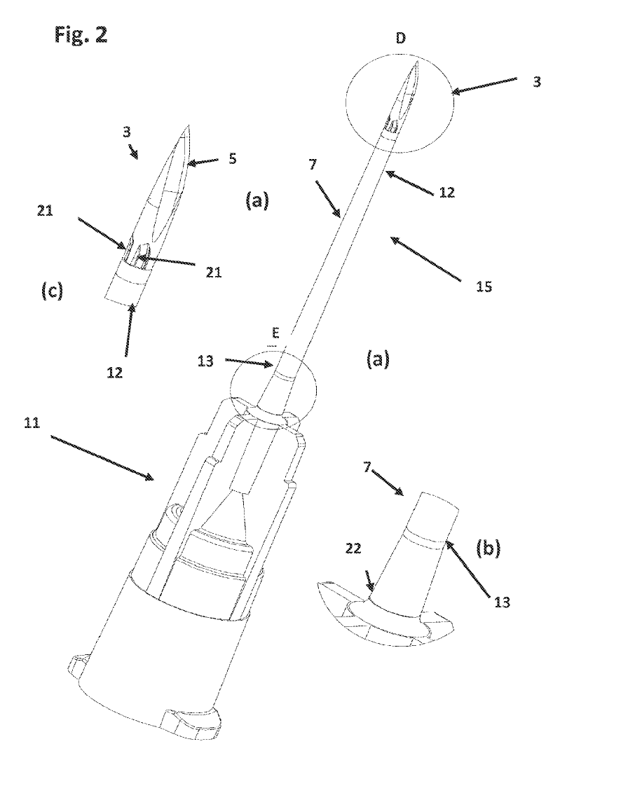

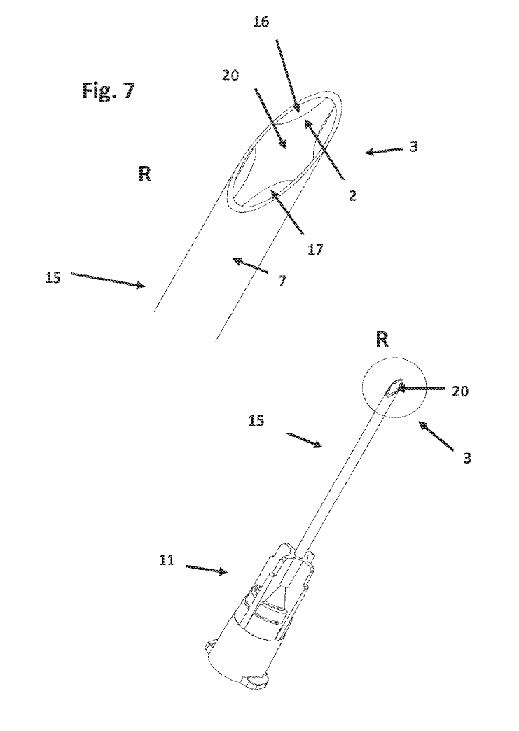

The sleeve 7 is a suitable length so that when engaged with the core needle 2, the distal portions of the channels 9 form apertures 16 for delivery of fluids from the conduits formed by the channels and the sleeve. Preferably, the apertures form side ports 21 as shown in FIG. 1 or FIG. 2c. Alternative embodiments may include apertures at the tip 3 of the needles as illustrated in FIG. 7. The scope of the invention includes many possibilities for the location of apertures or ports for fluid transmission other than the examples given herein. The shape of the apertures or ports will be determined by the shape of the channels 9, the engagement position of the sleeve 7 relative to both the channels and the core needle 2. Embodiments of the composite needle 15 incorporating side ports for fluid delivery most advantageously minimise the coring effect of tissue that is known to occur with ports in other configurations and other needles known in the art.

FIGS. 1b and 1c show more clearly the axial projections 10 and channels 9 at the proximal end 8 of a core needle or the distal end or tip 3 of the core needle 2 which are indicated by the circles C and B in FIG. 1a, respectively. Preferably, the tip end 3 of the core needle incorporates a cutting blade 5 and a sharp point 6 as shown in FIG. 1b for more efficient penetration of material or flesh to the site of fluid delivery. However, the tip 3 may have neither a cutting blade nor a sharp tip in some embodiments as shown in an alternative blunt embodiment of the tip 20 in FIG. 5.

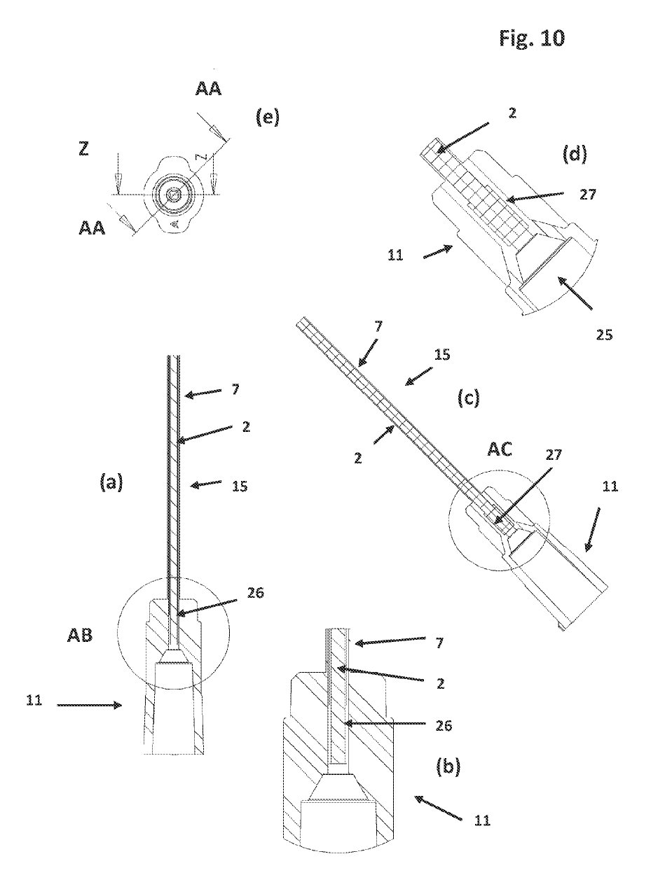

FIG. 1b shows the tip 3 of a core needle 2 incorporating engagement means 19 for engaging the distal end 12 of the sleeve 7. Similarly, FIG. 1 c shows the proximal end 8 of the needle 2 incorporating engagement means 22 for engaging the proximal end 13 of the sleeve 7. FIG. 1c also shows an embodiment having a radial raiser bore 18 for connecting the fluid conduits 17 with the hub 11 of the composite needle 15, enabling fluid to flow from the syringe barrel reservoir 25 (shown in FIG. 10) into the inside of hub 11 through the raiser bore 18 along longitudinal fluid bore and all the way to the needle point 3.

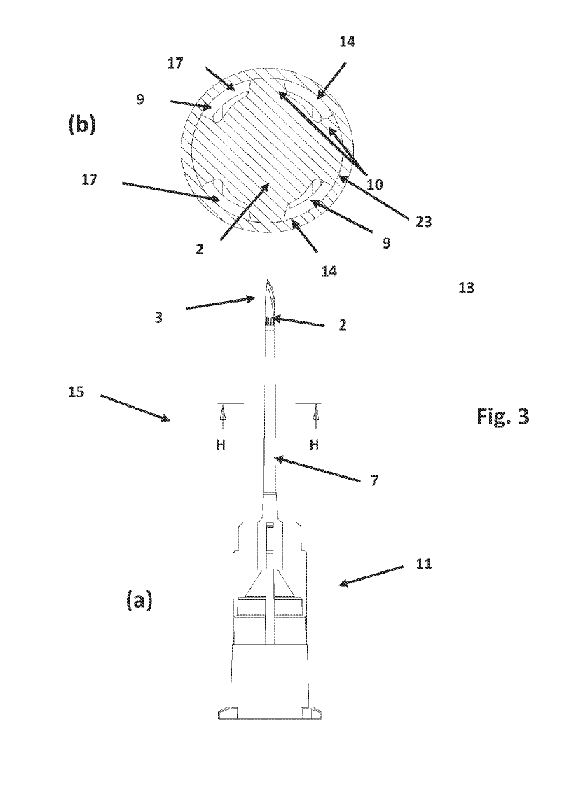

The engagement means may be a simple shoulder 19, 22 at both the distal (19) and proximal (22) ends of the core needle 2 which employs frictional engagement once in place. Such an engagement means most advantageously provides an external smooth surface for ease of penetration through materials or flesh of the assembled composite needle 15. The engagement of the sleeve 7 and core needle 2 may be achieved simply with the curved peripheral surface 23 of projections 10 of the core needle 2 as shown in FIG. 3.

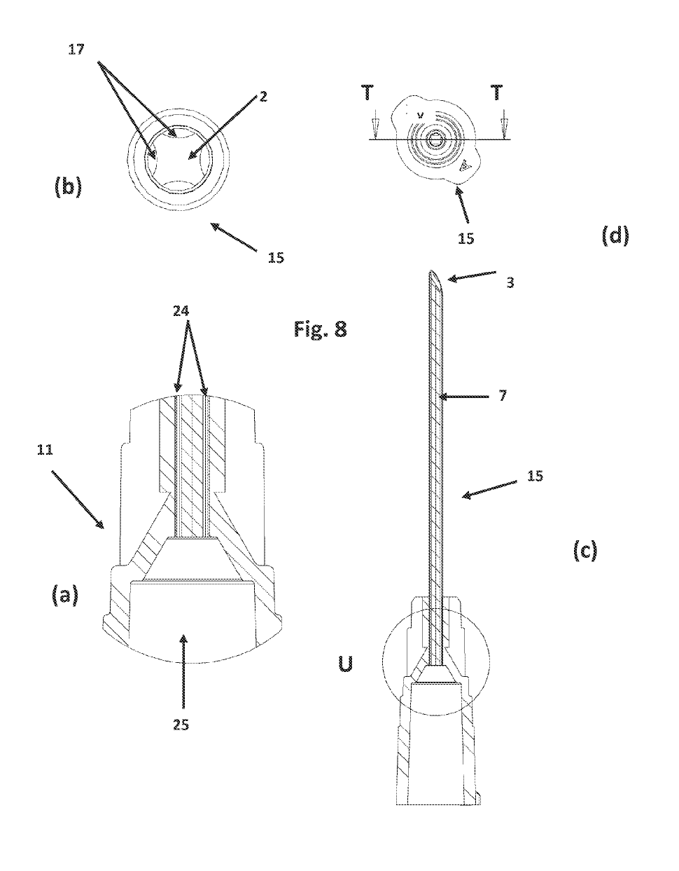

The core needle 2 incorporates a conduit for fluid transfer from a reservoir such as a syringe barrel 25 to through the hub 11 to the conduits 17 in the composite needle 15. One embodiment is shown in FIG. 8 where substantially linear hub conduits 24 are in direct fluid communication with the conduits 17 of the composite needle. Alternative embodiments for fluid transport include a raiser bore 18 for fluid communication between the fluid reservoir 25 of the syringe at the hub and each of the conduits 17 for fluid transfer with the composite needle to an injection site. A plurality of conduits requires a plurality of raiser bores in some embodiments.

FIG. 2 shows an embodiment of a composite needle 15 ready for use. The composite needle 15 comprises of the core needle within the sleeve 7. The tip end 3 of the core needle 2 is visible as is the proximal end 13 of the core needle. The sleeve 7 obscures most of the channels 9 of the core needle 2 and the core needle itself, except for the distal end. Because the sleeve 7 is slightly shorter than the length of the channels 9, the distal end forms open apertures 16 near the tip end 3 of the composite needle 15. Preferably, the apertures 16 are spaced from the tip end 3 of the needle and form side ports for fluid transfer. In these embodiments, there is less likelihood of coring of the penetrated material during penetration by the tip end. However, some embodiments of the invention may have apertures at the tip end 3 of the composite needle 15 such as that shown in FIG. 5. The proximal end 13 of the composite needle 15 does not have apertures so that fluid is conducted from a reservoir such as a syringe barrel into and along the conduit-forming channels to the apertures 16 at the tip end 3 of the composite needle.

FIG. 3 illustrates how the sleeve 7 and core needle 2 with channels 9 cooperate to form conduits 17 for fluid transmission. FIG. 3a shows in planar view an assembled composite needle 15 having a hub 11 a sleeve 7, and a tip 3. Only the tip 3 of the core needle 2 may be seen in the assembled composite needle. FIG. 3b shows a transverse section of the composite needle shown in FIG. 3a taken at the line designated H-H. The needle material in FIG. 3b is indicated by parallel lines within the surface curves 23. In this preferred embodiment, there are four axial projections 10 projecting longitudinally from the surface of the core needle 2, each axial projection 10 having a curved peripheral surface 23 for engaging the complementary inner surface 14 of the sleeve 7. Conduits 17 for fluid transmission are formed by the channels 9 in the core needle 2, the peripheral surfaces 23 of its axial projections 10 projection and inner surface 14 of the sleeve 7.

The engagement of the curved peripheral surfaces 23 is tight to ensure leak-free fluid transmission through the conduits 17. Preferably, frictional engagement between these surfaces will be adequate for engagement in many embodiments of the invention. Other engagement means such as the engagement shoulders 22, 19 may provide extra engagement force and keep the sleeve 7 in place. The shoulders at the distal end 19 and proximal end 22 ensure that the sleeve 7 cannot be moved along the axis of needle 15. Alternatively, the engagement may be with other means or any suitable process known in the art such as laser welding, heat shrinking, mechanical press fitting, or shrink wrapping. The most suitable engagement means or process will be determined by the materials used in the manufacture of the core pin and sleeve.

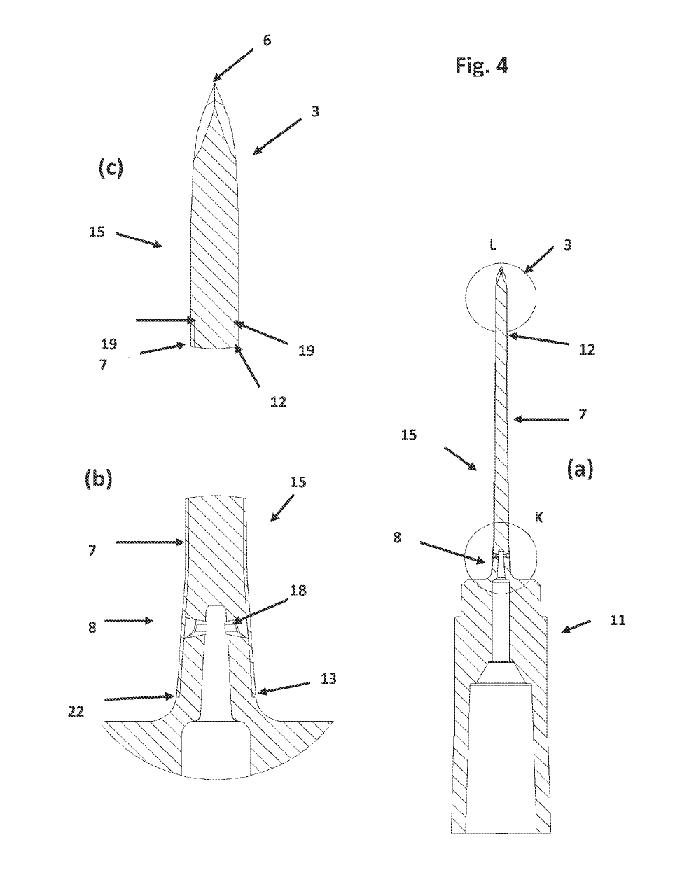

The sleeve engagement means is illustrated in longitudinal planar sections of a composite needle in FIG. 4 where the needle material is hatched with parallel lines. FIG. 4a shows a composite needle 15 having a proximal end at K and a distal end at L having a tip 3. The proximal end of the needle in the circle designated K is enlarged in FIG. 4b to show detail of the engagement of the proximal end of the sleeve 13 with the core needle 2 and the needle tip end at L is enlarged in FIG. 4c. In this embodiment the engagement means for engaging the sleeve 7 and core needle 2 is provided by the small shoulders 19 at each end of the sleeve 7 providing abutment surfaces to ensure that the sleeve 7 is frictionally engaged with the core needle 2. The shoulders 19, 22 at the engagement portions of the core needle are machined or moulded to ensure that the external surface of the composite needle is smooth to avoid an inadvertent tearing of tissue during penetration of the needle. The surface of the core needle 2 between the raiser bore 18 and the shoulder 22 form a sealing engagement with the inner surface of the sleeve 7 so that leaking can be prevented. FIG. 4c shows the engagement of the distal end 12 of the sleeve 7 with the shoulder 19 in the core needle 12 at the tip end 3 of the composite needle 15. The sleeve 7 is slid over the core needle 2 so either one of the sleeve or core needle must be made of resilient material to allow the sliding of the sleeve into place and engagement with the shoulder abutment surfaces. Other embodiments of the engagement means at the distal and proximal ends of the core needle and sleeve are possible, such as stepped shoulders, flanges, or the like. The engagement means chosen will depend on the materials used. Sleeves and core needles comprised of polymeric materials can incorporate many engagement means, limited only by the types of engagement means that can be moulded.

The most preferable materials used in the manufacture of sleeves and core needles are polymeric materials that are suitable for injection moulding. The scope of the invention includes a mould designed to form needles that are embodiments of the invention. The mould for core needle 2 does not require mechanical core pins. The channels 9 can be formed with conventional tooling technique. The mould split line for channels 9 of core needle 2 can be realised economically and without needing core pins or other special de-moulding technique.

FIG. 6 shows an exploded view of a further embodiment of the invention having a core needle 2 with a blunt tip 3 and a sleeve with an angled distal end 12 of the sleeve 7. FIG. 7 shows the composite needle 15 with the sleeve 7 in engaged position with the core needle. The blunt tip 3 of the core needle 2 can be seen in the circle at R. The core needle 2 and sleeve 7 in this embodiment are formed and engaged so that the distal end 12 of the sleeve 7 together form the tip 3 of the composite needle 15. In this embodiment, there is no engagement shoulder 19 or other frictional engagement means at the distal end of the sleeve. In this embodiment, the angle distal end 12 of the sleeve and the angled blunt end of the tip 3 of the core needle together form a tip which can penetrate tissue adequately with less coring than a flat blunt tip. Most advantageously, the core needle and sleeve of this embodiment and configuration are suitable for co-extrusion when the method of manufacture is injection moulding of polymeric materials.

FIG. 8 shows a longitudinal cross section of an alternative embodiment of a composite needle 15 having engaged core needle 2 and sleeve 7. FIG. 8a shows longitude conduits 24 in fluid communication with a reservoir portion of the hub 25. This embodiment of the invention provides an alternative configuration not requiring the raise bore 18, as shown in 1c and 4b, to connect the longitudinal conduits 24 with the hub reservoir 25. FIG. 8b shows the fluid conduits 17 and core needle 2 of composite needle 15.

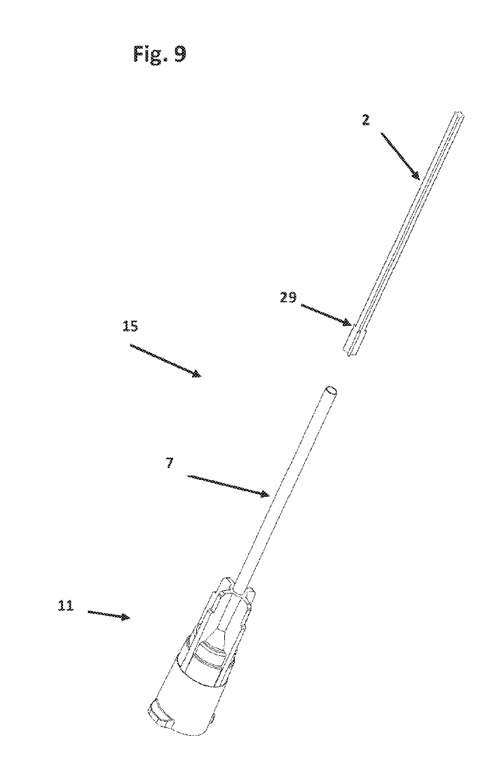

FIG. 9 shows an exploded view of a further embodiment of the invention having a sleeve 7 integral with a hub 11. The integration of the sleeve 7 and hub 11 in this embodiment most advantageously allows injection moulding of the integrated members as a single article. The inside of the hub may include engagement means for frictional engagement with the shoulders 29 of the core needle 2.

FIG. 10 shows a longitudinal cross section of an embodiment of a composite needle 15 having a sleeve 7 integrated with the hub 11 of a syringe. FIG. 10a shows a sleeve 7 engaged with a core pin 2, and the core pin 2 engaging the conduit of the hub 26. FIG. 10b is an enlarged section of AB of FIG. 10a showing greater details of the engagement of the sleeve 7 and core pin 2 with the hub internal surface 26 of the hub 7. FIG. 10c shows a further embodiment of a composite needle 15 including a core pin 2 and sleeve 7 engaged within a hub 11. In this embodiment, the proximal end 27 of the core pin 2 includes a flange for engagement with the inside of the hub 26. The portion at AC in FIG. 10c is more clearly illustrated in FIG. 10d.

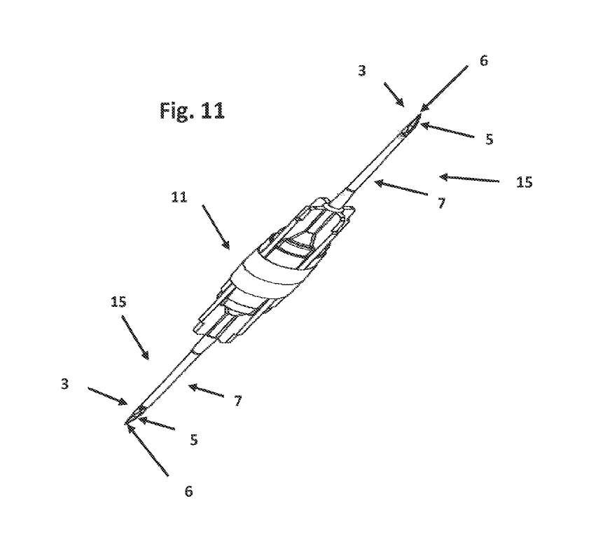

FIG. 11 shows a further embodiment of the invention wherein two composition needles 15 are conjoined at their hubs 11. In this embodiment, there is no reservoir 25 in fluid communication with the hubs. In this embodiment, fluids may be transferred from one container to another container through channels formed within the composite needles 15 as described herein. This embodiment most conveniently provides means to penetrate containers for fluid transfer between them.

FIG. 12 provides examples of other embodiments of a composite needle having spacers that are bump-like projections. Whereas the preferred embodiment includes projections projecting from the core needle, FIG. 12a illustrates in transverse section a composite needle wherein the projections 10 project from the inner surface of the sleeve 7 to form conduits 17 when engaged with the core needle 2. FIG. 12b illustrates in transverse section through a composite needle 15 wherein conduit forming spacers between the sleeve 7 and the core needle 2 are bump-like projections 26. The distribution of such projections may be regular, or irregular, on the core needle 2.

The figures herein illustrate embodiments of the invention having multiples conduit in a cannula with a sharp or pointed needle tip. The scope of the invention includes a cannula having multiple conduits therein but without a sharp or pointed needle tip.

1. A cannula comprising of:
a core needle having at least one longitudinal channel for fluid transport and an engagement means; and
a sleeve for engaging the core needle;
wherein a sealed conduit for fluid transport is formed when the sleeve is engaged with the said core needle.

2. The cannula of claim 1 wherein said channel is defined by spacers.

3. The cannula of either claim 1 or claim 2 where said spacers project from said core needle or said sleeve.

4. The cannula or any one of claims 1 to 3 wherein said spacers comprise of axial projections from the core needle.

5. The cannula of claim any one of claims 1 to 4, further comprising of engagement means for engaging said sleeve with said core needle.

6. The cannula of claim 5 wherein an engagement means comprises of a radial raiser bore connecting the longitude conduit with the needle hub.

7. The cannula of any one of claims 1 to 6, further comprising of a tip with a sharp point.

8. The cannula of any one of claims 1 to 7 further comprising of a tip with a sharp blade.

9. The cannula of any one of claims 1 to 8 further comprising of an aperture spaced from the tip end.

10. The cannula of any one of claims 1 to 9 further comprising of a traumatic tip with side port.

11. The cannula of any one of claims 1 to 10 wherein the sleeve is integrated in the hub.

12. The cannula of any one of claims 1 to 11 wherein at least one of the core needle or sleeve comprises of polymeric material.

13. A cannula according to any preceding claim having an outside diameter of 0.2 mm or bigger.

14. A mould for forming a cannula having a least one longitudinal channel in the peripheral surface of the cannula.

15. An apparatus to co-extrude a cannula according to any one of claims 1 to 11.

16. An apparatus to combine two materials with different properties to form a cannula according to any one of claims 1 to 13.

17. A method of manufacturing a cannula for fluid injection the steps of:
forming a core needle having channels in the peripheral surface;
forming a sleeve; and
engaging the sleeve with the core needle.
